(19) United States
(12) Patent Application Publication
(10) Pub. No.: US 2015/0126944 A1
(43) Pub. Date: May 7, 2015

(54) SYSTEM FOR APPLYING A SMALL QUANTITY OF ANTISEPTIC

(71) Applicant: Chlorhexidine Optimal Products For Skin LLC, Chesterton, IN (US)

(72) Inventor: Alexander A.L. Stemer, Chesterton, IN (US)

(73) Assignee: Chlorhexidine Optimal Products For Skin LLC, Chesterton, IN (US)

(21) Appl. No.: 14/074,427

(22) Filed: Nov. 7, 2013

(51) Int. Cl. A61M 35/00; A61K 31/155; A61J 1/14

(52) U.S. Cl. CPC A61M 35/003; A61J 1/1412; A61J 1/14; A61K 31/155; A61M 2210/04; A61J 2001/1418
USPC 604/290; 604/310

(57) ABSTRACT

The present invention comprises a system for applying small quantities of antiseptic such as a solution of chlorhexidine to a human patient. The system includes a container of 10-50 mL having an opening proximate the top end of the container and a closed end at the bottom. A dispenser for dispensing 0.05 to 0.2 mL of antiseptic through an opening of 0.8 to 4.8 mm is provided. A reclosable cap is further provided for sealing the top end of the container and retaining the antiseptic in the container. In a preferred embodiment, the dispenser comprises a roller ball of 1-5 mm which is rotatably mounted in the opening of the container, with a peripheral seal maintained around the opening for sealing the edges of the roller ball while allowing rotation thereof within the opening. This allows the antiseptic to remain on the ball so that the antiseptic may be dispensed to the patient in limited quantities without dripping from the dispenser.

### CROSS-REFERENCE TO RELATED APPLICATIONS

1. Technical Field

This invention relates to systems, methods and devices having particular utility in the field of therapeutic application of antiseptic to localized areas of skin surface. More particularly, the invention relates to articles and methods used for applying minute quantities of chlorhexidine solutions to an extremely small area on the human body.

2. Background of the Invention

Devices for application of antiseptic preparations on a patient's skin have included a wide variety of sponges and spray apparatus. These antiseptic solutions are generally applied with saturated sponges or held with forceps. Sometimes sponges with attached handles are provided in a plastic pouch containing enough liquid to saturate the sponges.

These techniques are often messy and offer little control over inadvertent dripping of the solution into areas where it is undesired. Particularly when applying antiseptic solution near the eyes, it is undesirable to have dripping or running of the antiseptic solution, especially when ingredients such as alcohol and iodine are used. A wide variety of applicators have been developed over the years for applying antiseptic solution but none of them to date have solved the above listed problems. Specifically, U.S. Pat. No. 6,672,784—Systems and methods for applying or dispensing surgical prep solution; U.S. Pat. No. 7,540,681—Systems and methods for applying or dispensing fluids such as surgical prep solution; EP2142067—Liquid applicators; U.S. Pat. No. 8,118,766—Skin antiseptic composition dispensers and methods of use are disclosed; U.S. Pat. No. 8,343,525—An antiseptic cleaning agent, generally, comprising chlorhexidine acetate and a solvent, such as an alcohol and/or water; WO2007076121—Surgical prep solution applicator; U.S. Pat. No. 5,829,976—Medicament-containing interproximal dental brush; U.S. Pat. No. 7,993,066—Liquid applicator and method for reducing the concentration of by-products from antiseptic; LACER Chlorhexidine; CHLORHEXIL 0.20% spray; Chlorhexidine and Zinc Oral Spray 8 oz bottle; Gold Vet Sptd-chlorhexidine Spray For Dogs And Cats; Chlorhexidine cleansing spray; Chlorhexidine Gluconate 2% and 70% isopropyl alcohol surgical swab; q2° Q•Care® Rx Oral Cleansing and Suctioning System with 3M™ Peridex™ (Chlorhexidine Gluconate 0.12%) Oral Rinse and Thumb Port Tools; Corsodyl spray (chlorhexidine); Petrodex Dental Spray for Dogs & Cats, 4 oz; Spray désinfectant chlorhexidine; ChlorHex 2 X Topical Spray—with 4% Chlorhexidine—8 oz; DENTAID PERIO-AID 0.12% chlorhexidine mouth spray 50 m; and Package/Label Principal Display Panel.

While these described products provide considerable advances, they do not prevent dripping of the antiseptic solution in localized areas and are not suitable for applying small quantities of antiseptic to minute areas of the skin.

### BRIEF SUMMARY OF THE INVENTION

The present invention provides systems and methods for applying or dispensing antiseptic solutions in small quantities for application to a localized area. Among the potential advantages of the systems and methods of the invention are the ability to dispense antiseptics, particularly chlorhexidine solutions, substantially drip-free. The invention is particularly useful in treating small lesions, especially near the eyes.

The system for applying small quantities of antiseptic comprises a container of 10-15 mL, an opening proximate to the top end of said container and a closed end at the bottom of said container. A dispenser for dispensing 0.05 to 0.2 mL of antiseptic is provided having an opening of 0.8 to 4.8 mm. A reclosable cap may be affixed to the top end of the container for sealing the top end of the container and retaining the antiseptic inside container. In a preferred embodiment the dispenser further comprises a roller ball of 1-5 mm rotatably mounted in said opening of said container and a peripheral seal in said opening for rotatably sealing the edges of said roller ball within said opening while allowing said antiseptic to remain on said ball, thereby allowing dispensing of said antiseptic without dripping from said dispenser.

In a preferred embodiment the antiseptic comprises 2-4% chlorhexidine suspended in a solution from the group selected from alcohol or water. The solution may comprise a non-alcoholic solution or, in a preferred embodiment, comprises chlorhexidine acetate wherein the chlorhexidine acetate constitutes about 0.01% to about 10% of the solution and the remainder of the solution comprises isopropanol and ethanol wherein the ratio of isopropanol to ethanol is from about 1:1000 to 1000:1. The present system includes a container which is substantially tubular in shape and has a diameter of ½ inch±¼ inch and a length of between 2 and 6 inches with an interior volume of 10 mL±20 ml. In an alternative embodiment of the invention the antiseptic is in the form of a gel and the dispenser comprises a threaded shaft which extends through the center of the tube and a rotatable wheel proximate the base of the shaft with the gel being substantially solid and disposed on a platform threadably mounted on said shaft and operatively connected to said shaft so that rotation of said shaft effects upper movement of said platform towards said opening, thereby forcing said gel through said opening in controlled quantities. The system may include antiseptic which is also in the form of a semiliquid state.

Included in the invention is a dispenser for minute quantities of antiseptic to a limited site on the human body. The dispenser includes a hollow tube sealed at a first end with a roller ball of 1-5 mm rotatably mounted at the second end of the tube, in an opening of the tube. A mechanism for sealing the edges of the roller ball within the opening is provided, but still allows rotation of the roller ball and passage of antiseptic on the surface of the roller ball from inside the container to the outside of the container on the roller ball after it has been rotated. A cap is further provided for sealing the second end of the tube when the dispenser is not in use.

While there are a wide variety of applicators for antiseptics, as noted above, to the best of our knowledge no one has used a roller ball for applying an antiseptic and in particular no one has used a roller ball having a small diameter so as to apply a limited quantity of antiseptic over a small area on the patient's skin. While roller balls are known for application of deodorant, the use of an extremely small roller ball is novel and unique, and provides unexpected advantages in limiting spillage and an ability to apply a limited quantity of antiseptic on an extremely small area of the patient. While it is recognized that merely changing the size of a device is not patentable, in the present case the reduction in size provides unexpected advantages not found in the prior art.

In a preferred embodiment, the antiseptic comprises 2% to 4% chlorhexidine suspended in a solution. The tube is constructed of polyethylene, and the roller ball is constructed of polycarbonate. The opening for the dispenser is 0.8 mm to 4.8 mm, depending on the size of the ball, which allows rotation of the ball with antiseptic adhering thereto. In an alternative embodiment of the invention the container may be a bottle which is constructed of glass, polyethylene, polypropylene, polycarbonate or acrylic. Similarly, the rotatable ball may also be constructed of polyethylene.

The present invention includes a method of applying a small quantity of antiseptic to a small area of the human body without spillage or excess. The present invention applies effective but safe doses of potentially toxic substances, specifically chlorhexidine, to all areas of the body, but specifically is concerned with treatment of facial lesions, which pose a safety risk due to the danger of eye contact. The method comprises providing a bottle of antiseptic having a roller ball to 1-5 mm in size at the upper end of the bottle and a closure over the roller ball for selective sealing of the bottle as required. The method includes the step of removing the closure, which in a preferred embodiment is a threaded cap, inverting the bottle and rolling the antiseptic onto the small area of the human body. The antiseptic preferably comprises chlorhexidine in a 2-4% solution which has sufficient viscosity to avoid dripping or running on the patient's skin. Following application of the antiseptic, the roller ball and bottle are removed from the patient and the bottle is returned to an upright position and closed using the closure cap.

An additional feature of the invention is that the bottle and applicator may be used for multiple applications of small doses of antiseptic to the face of the human, as is required. The applicator, again, comprises a container of 10-60 mL with an opening proximate the top of the container, the container being closed at the bottom end thereof. The dispenser for dispensing 0.05 to 0.2 mL of antiseptic is provided at the top of the container, which has an opening into the container of 0.8 mm to 4.8 mm, depending on the size of the ball. A reclosable cap is further provided for sealing the top of the container and retaining the antiseptic in the container after use. The container may then be reopened as required for new applications of the antiseptic. The antiseptic maintains itself in a sterile condition. The system in an alternative embodiment may comprise a flexible plastic tube such as a toothpaste tube which is closed at one end, when applying a gel to the desired area of the human body. A suitable material for the container may be a polymer/aluminum foil laminate such as a polyolefin/foil laminate. In this embodiment, the container is squeezed, which dispenses the gel antiseptic onto the patient. The use of a collapsible tube assists in avoiding dripping.

It may also be preferred that at least the exterior surfaces of the container be sterile such that the applicator system can be used in an aseptic manner to apply the antiseptic solution. This may require that the dispenser be provided in a sterile package.

These and other features and advantages of the present invention are described below in connection with the various illustrative embodiments of the invention.

In a preferred embodiment, the roller ball applicator of the present invention has a rigid spherical ball located in the bottle opening adjacent a neck down portion for forming a basket for retaining the ball. The basket portion of the bottle is generally spherical and slightly larger than the ball and the opening is formed to be of a diameter slightly less than the ball, i.e., less than 2-6 mm depending on the ball size. By choosing a flexible material for the basket, the rigid ball is generally capable of being forced into the basket and is thereafter retained by the smaller diameter opening of the bottle. In those embodiments in which a rigid bottle is required, polyvinyl chloride or polyethylene terephthalate better known as PBC and PET may be used. In a preferred embodiment the container may have a glass or crystal clarity for viewing the contents contained therein. A circular retaining ring having a lower surface is provided for mating engagement with the upper edge surface of the bottle opening and is positioned such that the lower surface of the circular retaining ring is in contact with the upper edge surface of the bottle opening. The circular retaining ring is then welded to the bottle opening upper edge by ultrasonic or other heat sealing means.

The roll-on applicator of the invention may also comprise an open top bottle of rigid clear material having a necked down portion adjacent the upper end thereof and wall structure forming a ball retaining enclosure at the open bottle top. The wall structure comprises a radially outwardly extending inner wall connecting to an upwardly extending inner wall that is substantially parallel to the axis of the bottle and terminates in an upper edge surface of the bottle opening. A circular retaining ring having a lower surface for mating engagement with the upper edge surface of the bottle opening and an inner wall substantially parallel to the axis of the retaining ring and terminating in an inwardly extending lip at the opening is provided, the ring lower surface being affixed to the bottle upper edge surface such that the bottle inner wall is substantially parallel to the bottle axis and is in alignment with the ring inner wall to form a cylindrical cavity. An inwardly extending lip projects from the ring upper edge surface to produce an opening of lesser diameter than the cylindrical cavity and a rigid spherical ball having a diameter greater than the opening produced by the lip is disposed within the cylindrical cavity such that the upper edge surface of the bottle is in direct contact with the lower surface of the circular retaining ring with the ring affixed to the bottle. By ultrasonically welding the ring to the bottle, the bottle edge surface and the ring lower surface are sealing having no substance therebetween such as adhesive.

The roll-on applicator of the invention comprises an open top bottle of rigid clear material having a necked down portion adjacent the upper end thereof and wall structure forming a ball retaining enclosure at the open bottle top. The wall structure comprises a radially outwardly extending inner wall connecting to an upwardly extending inner wall which is substantially parallel to the axis of the bottle and terminates in an upper edge surface of the bottle opening. A circular retaining ring having a lower surface for mating engagement with the upper edge surface of the bottle opening and an inner wall substantially parallel to the axis of the retaining ring and terminating in an inwardly extending lip at the opening is provided, the ring lower surface being affixed to the bottle upper edge surface such that the bottle inner wall is substantially parallel to the bottle axis and is in alignment with the ring inner wall to form a cylindrical cavity. An inwardly extending lip projects from the ring upper edge surface to produce an opening of lesser diameter than the cylindrical cavity and a rigid spherical ball having a diameter greater than the opening produced by the lip is disposed within the cylindrical cavity such that the upper edge surface of the bottle is in direct contact with the lower surface of the circular retaining ring with the ring affixed to the bottle. By ultrasonically welding the ring to the bottle, the bottle edge surface and the ring lower surface are sealing having no substance therebetween such as adhesive. It should be noted that in contrast to prior roll-on devices, the present invention comprises an opening of from 0.8 to 4.8 mm and the roller ball is 1-5 mm. Thus, a minute quantity of antiseptic may be applied unlike a conventional roller ball applicators for deodorant.

### BRIEF DESCRIPTION OF THE SEVERAL VIEWS OF THE DRAWING

FIG. 1 of the drawings is a front perspective exploded view of the roller ball dispenser of the present invention.

FIG. 2 of the drawings is a front perspective view of the roller ball apparatus of the present invention in an assembled configuration.

FIG. 3 of the drawings is a side cutaway view of the roller ball of the present invention mounted in the dispenser of the container of the present invention with the roller ball being disposed within the dispenser of the container.

FIG. 4 of the drawings is a front perspective view of an alternative embodiment of the antiseptic dispenser of FIG. 1 showing a squeezable tube for dispensing antiseptic.

FIG. 5 of the drawings is a front perspective view exploded of an additional alternative embodiment of the antiseptic dispenser of FIG. 1 showing a tube having a push-up mechanism for dispensing semisolid antiseptic through the top of the dispenser.

FIG. 6 of the drawing is a front perspective view of the push-up dispenser of FIG. 5 showing assembled view.

FIG. 7A of the drawings is a front perspective exploded view of an additional alternative embodiment of the invention in which a container in the form of a collapsible tube is provided having a sealed bottom end an open top end of approximately 2 mm, and a cap sealing said open top end. Antiseptic in the form of a gel or in a semiliquid state may be dispensed through said opening by squeezing of said tube.

FIG. 7B of the drawings is a front perspective view of the alternative embodiment of the invention shown in FIG. 7A.

FIG. 8 of the drawings is an alternative embodiment in which the container is a bottle used in an embodiment of a method of the present invention for applying minute amounts of antiseptic, particularly chlorhexidine, to a localized area of human skin.

### DETAILED DESCRIPTION OF THE INVENTION

While the present invention is shown in the following drawings it is understood that the invention is not limited thereto except and so far as those who have the disclosure before they are able to make modifications and variations thereof without departing from the scope of the invention.

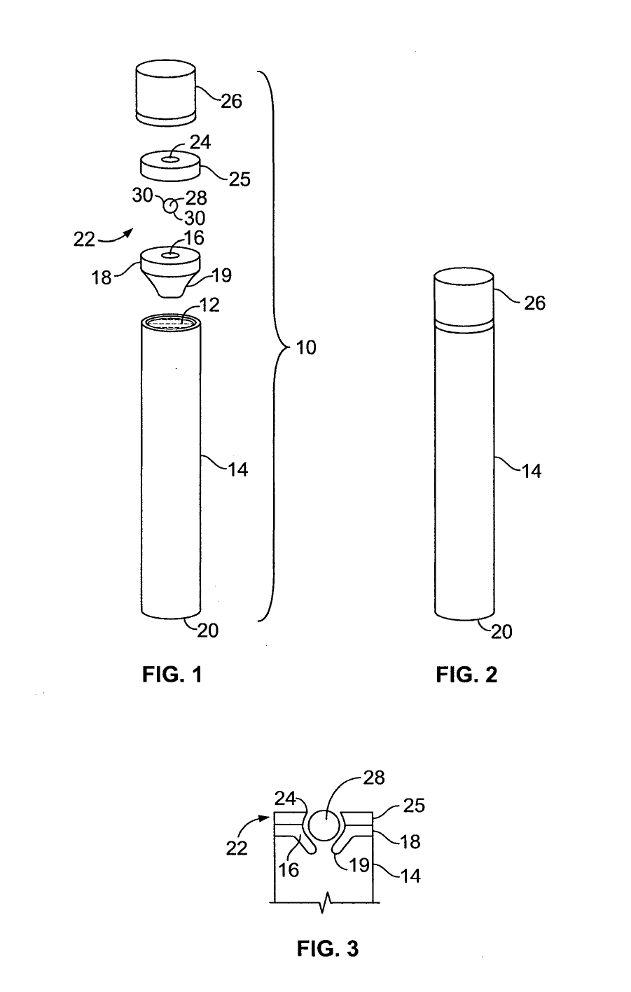

As seen in FIG. 1 of the drawings a system 10 for applying small quantities of antiseptic 12 is disclosed. The system includes a container 14 10-60 mL in size. An opening 16 proximate the top end 18 of container 14 is shown. Container 14 is closed at its bottom end 20. A dispenser 22 is provided proximate the top end 18 of container 14 for dispensing 0.05 to 0.2 mL of antiseptic 12. Dispenser 22 has an opening 24 which is 0.8 to 4.8 mm in diameter, depending on the size of the ball, so as to limit the amount of antiseptic 12 being dispensed. A reclosable cap 26 is provided for sealing top end 18 of said container 14 and for retaining antiseptic 12 within said container when said system is not in use. In a preferred embodiment, a roller ball 28 may be rotatably mounted in a basket 19 located proximate the top end 18.

Dispenser 22 comprises a roller ball 28 of 1-5 mm rotatably mounted in opening 16 and a peripheral seal 25 in said opening 16 for sealing the edges 30 of said roller ball 28 while allowing rotation thereof within said opening 16, thereby allowing said antiseptic 12 to remain on said roller ball 28 thereby allowing dispensing of said antiseptic 12 without dripping from said dispenser 22.

While there are a wide variety of applicators for antiseptics, as noted above, to the best of our knowledge no one has used a roller ball for applying an antiseptic and in particular no one has used a roller ball having a small diameter so as to apply a limited quantity of antiseptic over a small area on the patient's skin. While roller balls are known for application of deodorant, the use of an extremely small roller ball is novel, unique, and provides unexpected advantages in limiting spillage and an ability to apply a limited quantity of antiseptic on an extremely small area of the patient. It is recognized that merely changing the size of a device is not patentable. In the present case, the reduction in size provides unexpected advantages not found in the prior art.

In a preferred embodiment the antiseptic 12 comprises 2-4% chlorhexidine suspended in a solution comprising one of the group of water or isopropanol.

In a preferred embodiment, the container 14 is substantially tubular in shape, having a diameter of ½ inch±¼ inch and a length of 2-4 inches with an internal volume of 10-60 mL. In one embodiment, the diameter is ¼ inch-½ inch, the length is 4 inches, and the internal volume is approximately 10 mL.

Referring to FIG. 3, a cross section of a portion of the dispenser 22 attached to the top end 18 of the container 14 from FIG. 1 is shown. As shown, in a preferred embodiment the roller ball 28 may be rotatably mounted in a basket 19 at the top end 18 of the container 14. The peripheral seal 25 of the dispenser 22 allows the roller ball 28 to rotate freely while coated in antiseptic 12. The basket 19 permits the antiseptic 12 to come into contact with the roller ball 28 while retaining the roller ball 28 within the dispenser 22.

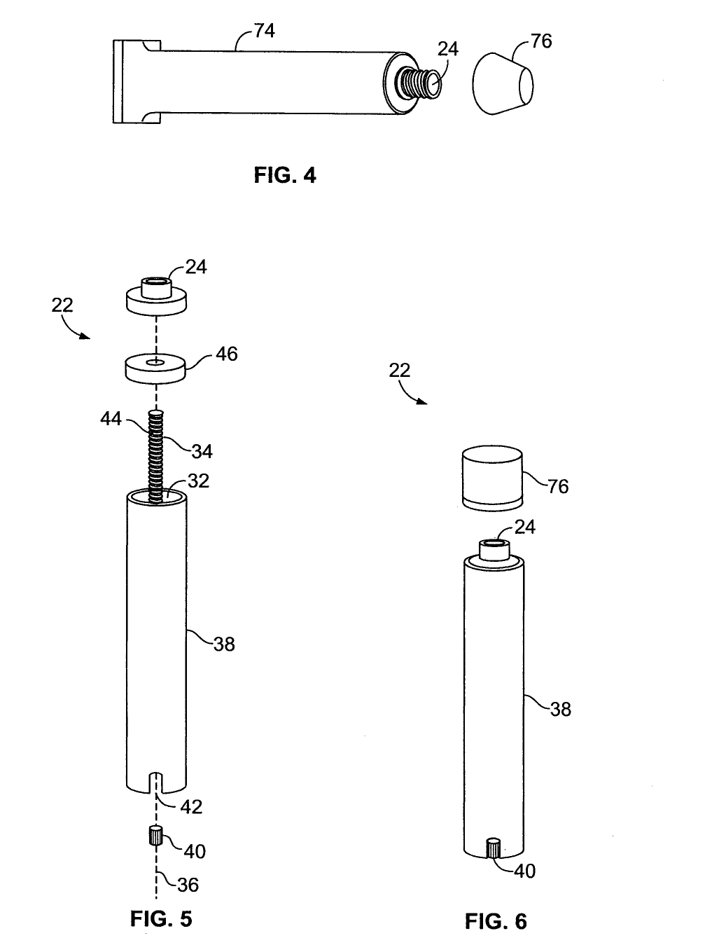

As seen in FIG. 4, in an alternative embodiment the container 14 may comprise a flexible plastic tube 74 which is squeezable to dispense an antiseptic through the opening 24 of the tube. In this alternative embodiment, the antiseptic is in the form of a gel, like toothpaste. The tube may be sealed using a cap 76 to prevent accidental disbursement of the antiseptic when the tube is not in use.

In an alternative embodiment, antiseptic 12 as seen in FIGS. 5 & 6 may come in the form of a gel, and dispenser 22 comprises a threaded shaft 34 extending longitudinally through the center 36 of tube 38. Referring to FIG. 5, an exploded perspective view is shown. A rotatable wheel 40 is attached to said shaft proximate the base 42 of the shaft 34. A platform 46 is threadedly mounted on shaft 34 and is operatively connected to said threading 44 on said shaft 34 so that the rotation of said shaft 34 effects upward movement of said platform 46 towards said opening 24, thereby forcing said gel 32 through said opening 24 in controlled quantities such as 0.05 to 0.2 mL. In an alternative embodiment, antiseptic 12 may be suspended in a semiliquid state. In a preferred embodiment, antiseptic 12 comprises 2-4% chlorhexidine suspended in a solution. In a preferred embodiment, container 14 comprises a tube 38. Referring to FIG. 6, the tube 38 optionally may be sealed using a cap 76.

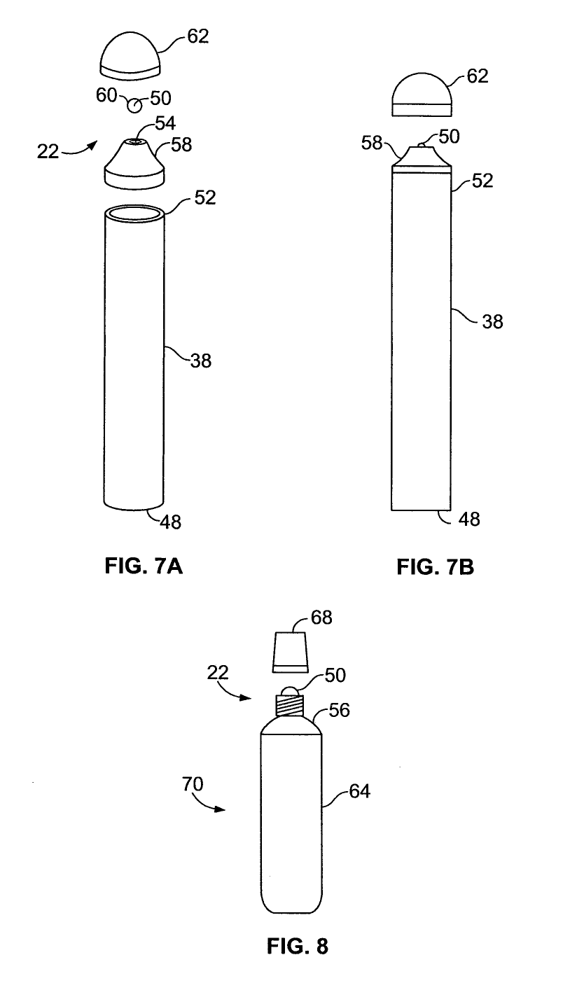

As seen in FIGS. 7A and 7B, in a preferred embodiment container 14 comprises a tube 38 sealed at a first or distal end 48 having a roller ball 50 rotatably mounted at the second or proximal end 52 of said tube 38 in an opening 54 of said tube 38. A mechanism 58 is provided for sealing the edges 60 of said roller ball 50 within said opening 54. A cap 62 seals said second end 52 of said tube 38 when said dispenser 22 is not in use. The tube 38 is preferably constructed of polyethylene, and the roller ball 50 is preferably constructed of polycarbonate. However, other commonly known polymers may be used, such as polypropylene or polyethylene terephthalate, glass, polycarbonate or acrylic. The opening 54 in said tube 38 is 0.8 mm+4.8 mm, although in some embodiments it may be more or less.

As further seen in FIG. 8, an additional feature of the invention is a method for applying a small quantity of antiseptic 12 to a small area of the human body without spillage or excess using an applicator 70. The method comprises providing a bottle or container 64 of antiseptic 12 having a roller ball 50 of 1-5 mm in diameter at the upper end 66 of said bottle 64 and a closure 68 over the roller ball 50. The closure 68 is removed, and the bottle is inverted. The antiseptic 12 thereby contacts the roller ball. When the roller ball contacts the human skin and is rolled thereon, antiseptic 12 is deposited onto a small area of the human body, since the ball itself is only 2 mm in diameter. The bottle 64 is then withdrawn from the skin of the patient, and the bottle is closed using closure 68.

Referring again to FIG. 1, in a preferred embodiment, the skin antiseptic composition exits the applicator system 10 through the dispensing opening 24 under force of gravity and deposits minute quantities of said skin antiseptic on the patient. A cap 26 may then be used for sealing the dispensing apparatus and the container. In a preferred embodiment, the dispensing apparatus 22 comprises a roller ball 28 affixed to the proximal end 18 of the container 14 and a sealing mechanism preferably a basket 19 for rotatably containing the roller ball 28. While chlorhexidine is the preferred antiseptic, other non-alcoholic biocidal agents may also be used. In the preferred embodiment the biocidal agent is chlorhexidine acetate, which comprises about 0.01% to about 10% of a solution by weight, and more than 90% of the antiseptic solution comprises an alcohol comprising isopropanol and ethynol wherein a ratio of the isopropanol to ethynol is from 1:1000 to 1000:1.

1. A system for applying small quantities of antiseptic comprising:
a container of 10-60 mL;
an opening of 0.8 to 4.8 mm proximate the top end of said container, said container being closed at the bottom end;
a dispenser proximate said opening for dispensing 0.05 to 0.2 mL of chlorhexidine antiseptic; and
a reclosable cap for sealing said top of said container and retaining said antiseptic in said container.

2. The system of claim 1, wherein said dispenser comprises a roller ball of 1-5 mm rotatably mounted in said opening and a peripheral seal in said opening for sealing the edges of said roller ball while allowing rotation thereof within said opening while allowing said antiseptic to remain on said ball, thereof allowing dispensing of said antiseptic without dripping from said dispenser.

3. The system of claim 1, wherein said antiseptic comprises 2% chlorhexidine suspended in a solution comprising an alcohol comprising isopropanol and ethanol, wherein a ratio of the isopropanol to ethanol is from about 1:1000 to 1000:1.

4. The system of claim 1, wherein said container is substantially tubular having a diameter of ½ inch and a length of 2-3 inches±1 with an interior volume of 10 mL+20.

5. The system of claim 4, wherein said antiseptic is in the form of a gel and said dispenser comprises a threaded shaft extending through the center of said tube, a rotatable wheel proximate the base of the shaft, a platform threaded mounted on said shaft and operating connected to said shaft so that rotation of said shaft effects movement of said platform towards said opening, thereby forcing said gel through said opening in controlled quantities.

6. The system of claim 1, wherein said antiseptic is in a semiliquid state.

7. A dispenser for minute quantities of antiseptic to a limited site, said dispenser comprising:
a tube sealed at a first end;
a roller ball of 1-5 mm rotatably mounted at the second end of said tube, in an opening of said tube;
means for sealing the edges of said roller ball under said opening; and
a cap for sealing said second end of said tube when said dispenser is not in use.

8. The dispenser of claim 7, wherein said antiseptic comprises 2 to 4% chlorhexidine suspended in a solution.

9. The dispenser of claim 7, wherein said tube is constructed of polyethylene and said roller ball is constructed of polycarbonate.

10. The dispenser of claim 7, wherein said opening is 0.8 mm to 4.8 mm.

11. A roller ball applicator for minute presentation of antiseptic, said applicator comprising:
a bottle;
a roller ball rotatably mounted in the top of said bottle;
sealing means disposed around said roller ball;
antiseptic within said bottle; and
cap means for selectively and sealing said applicator.

12. The roller ball applicator of claim 11, wherein said roller ball is 1-5 mm in diameter.

13. The roller ball applicator of claim 11, wherein said bottle is construed from the group consisting of glass, polyethylne, polypropylene, polycarbonate, polyethylene terephthalate, Polyvinylidene Chloride or acrylic.

14. The roller ball applicator of claim 11, wherein said antiseptic comprises 2% chlorhexidine suspended in a solution comprising an alcohol comprising isopropanol and ethanol, wherein a ratio of the isopropanol to ethanol is from about 1:1000 to 1000:1.

15. The roller ball applicator of claim 11, wherein said bottle is substantially tubular having a diameter of ½ inch and a length of 2-3 inches with an interior volume of 10 mL to 30 ml.

16. The roller ball applicator of claim 11, wherein said antiseptic is in a semiliquid state.

17. The roller ball applicator of claim 11, wherein said antiseptic comprises 2 to 4% chlorhexidine suspended in a solution.

18. The roller ball applicator of claim 11, wherein said roller ball is mounted in an opening of 0.8 mm to 4.8 mm.

19. The roller ball applicator of claim 11, wherein said antiseptic comprises chlorhexidine.

20. A method of applying a small quantity of antiseptic to a small area of the human body without spillage or excess, said method comprising:
providing a bottle of antiseptic having a roller ball of 1-5 mm at the upper end of the bottle and a closure over the roller ball;
removing the closure;
inverting the bottle and rolling the antiseptic onto the small area of the human body;
withdrawing the bottle;
returning the bottle to an upright position; and
closing said closure.

21. A multiple-use applicator for delivering small doses of antiseptic to the face of a human, comprising:
a container of 10-30 mL;
an opening proximate the top end of said container, said container being closed at the bottom end;
a dispenser opening of 0.8 to 4.8 mm for dispensing 0.05-0.2 mL of chlorhexidine antiseptic; and
a reclosable cap for sealing said top of said container and retaining said antiseptic in said container; and
wherein said antiseptic is in a sterile condition.

22. The system of claim 1, in which said container comprises a flexible plastic tube.

23. The system of claim 23, in which said container is squeezable to dispense said antiseptic and said antiseptic is in the form of a gel.

24. A skin antiseptic composition dispenser comprising:
a container defining an interior volume wherein the container comprises a distal end and a proximal end disposed along a longitudinal axis.
a skin antiseptic composition located within the interior volume of the container;
at least one dispensing opening proximate the first end of the container;
a dispensing apparatus closing the at least one dispensing opening;
an applicator attached to the dispensing apparatus at the proximal end of the container, wherein the dispensing opening is in fluid communication with the applicator;
said dispensing opening being between 0.8 and 4.8 mm in diameter;
wherein the skin antiseptic composition exits the applicator through the dispensing opening under the force of gravity and deposits minute quantities of said skin antiseptic on a patient; and
a cap for sealing said dispensing means and container.

25. The skin antiseptic composition dispenser of claim 24, in which said dispensing apparatus comprises a roller ball rotatably affixed to said proximal end of said container, including sealing means for sealing said roller ball to said opening of said container but allowing rotation of said roller ball.

26. The skin antiseptic composition dispenser of claim 24, in which said antiseptic comprises a non-alcoholic biocidal agent.

27. The skin antiseptic dispensing apparatus of claim 24, in which said skin antiseptic composition comprises chlorhexidine acetate, wherein the chlorhexidine acetate comprises about 0.01% to about 10% of the solution by weight, and more than about 90% of the antiseptic solution comprises an alcohol comprising isopropanol and ethanol, wherein a ratio of the isopropanol to ethanol is from about 1:1000 to 1000:1.

28. A method of applying a small quantity of antiseptic to a small area of the human body without spillage or excess, said method comprising:
providing a container of antiseptic having an opening of 0.8 to 4.8 mm at the upper end of the container and a closure over said opening;
removing the closure;
inverting the container and applying the antiseptic onto the small area of the human body;
withdrawing the container;
returning the container to an upright position; and
closing said closure.

29. The method of claim 28 wherein said antiseptic comprises chlorhexidine.

30. The method of claim 28 and further comprising application of a small quantity of antiseptic to small lesions, including lesions near the eyes.

31. A method of applying a small quantity of antiseptic to a small area of the human body without spillage or excess, said method comprising:
providing a container of antiseptic having an opening at the upper end of the container and a closure over said opening;
removing the closure;
inverting the container and applying 0.05-0.2 mL of the antiseptic onto the small area of the human body without spillage or excess;
withdrawing the container;
returning the container to an upright position; and
closing said closure.
